(19) United States
(12) Patent Application Publication
(10) Pub. No.: US 2012/0308541 A1
(43) Pub. Date: Dec. 6, 2012

(54) PHARMACEUTICAL COMPOSITION FOR TREATING THE GASTROINTESTINAL TRACT

(76) Inventor: Eliezer Smoler, Gan Yavne (IL)

(21) Appl. No.: 13/578,073

(22) PCT Filed: Feb. 9, 2011
(86) PCT No.: PCT/IL2011/000139

§ 371 Date: Aug. 9, 2012

(51) Int. Cl. A61K 38/43; A61K 33/44; A61P 3/00; A61K 33/00; A61P 1/04; A61P 1/12; A61K 38/54; A61K 33/06

(52) U.S. Cl. 424/94.1; 424/94.2; 424/600; 424/684; 424/724

(57) ABSTRACT

Provided are pharmaceutical compositions for the treatment or prophylaxis of the gastrointestinal tract. One of the pharmaceutical composition includes co-particles of an adsorbent, and one or more carbohydrates. This pharmaceutical composition may be used, for example, in the treatment or prophylaxis of diarrhea, for adsorbing digestive tract microorganisms, for enhancing weight gain, or for enhancing milk production in a mammal. Another pharmaceutical composition can include one or more enzymes and a pH buffer system, wherein the pH buffer system causes an alkalinization of at least a portion of a gastrointestinal tract. This pharmaceutical composition may be used, for example, for increasing milk production in a mammal.

### FIELD OF THE INVENTION

This invention relates to dietary supplements, and such supplements for treating the gastrointestinal tract of a human or animal.

### BACKGROUND OF THE INVENTION

Activated carbon, also called activated charcoal or activated coal is a form of carbon that has been processed to make it extremely porous and thus to have a very large surface area available for adsorption or chemical reactions. Active charcoal has long been known as a medicament endowed with the ability to adsorb toxins and gases, and as such to facilitate intestinal detoxification.

Tablets or capsules of activated charcoal have been used as an over-the-counter drug to treat diarrhea, indigestion, and flatulence. There is some evidence of its effectiveness as a treatment for irritable bowel syndrome (IBS).

U.S. Pat. No. 7,258,879 to Hodge et al discloses a pet food product comprising vegetable charcoal and other ingredients for reducing flatulence odor in pets.

International Patent Publication WO0149128 discloses a dietary supplement for animals comprising one or more enzymes and a physiologically acceptable carrier.

### SUMMARY OF THE INVENTION

In one of its aspects, the present invention provides a pharmaceutical composition for treating a gastrointestinal tract of a human or animal. The pharmaceutical composition, in accordance with this aspect of the invention, comprises co-particles of an adsorbent and one or more carbohydrates. The pharmaceutical composition in accordance with this aspect of the invention may be used to reduce or prevent cases of diarrhea and other digestive disorders in ruminating and non-ruminating animals, for example, disorders caused by toxins present in the animal feed or toxins released into the digestive tract by endomicroorganisms. The inventor has found that the pharmaceutical composition of the invention tends to produce better results than adsorbent alone.

The adsorbent may be, for example, active charcoal, silica-gel, zeolites, aluminum silicate, or a synthetic or naturally occurring resin. If the absorbent is active charcoal, the active charcoal may be from a plant source. The pharmaceutical composition may be prepared by mixing a powder of the adsorbent with a powder of one or more carbohydrates under conditions that allow the particles of the two powders to adhere to one another to form co-particles having an adsorbent moiety and a carbohydrate moiety. The composition is preferably maintained in a dry state at all times. The inventors have found, for example, that a composition of the invention may be prepared in which the adsorbent is active charcoal and the carbohydrate is dextrose by mixing powders of the charcoal and the dextrose in a mixer or blender at ambient temperature. Under these conditions, the two particle types spontaneously adhere to each other to form co-particles.

The ratio of adsorbent to carbohydrates may be, for example, from 1:1 to 9:1. 80:20.

For prophylactic purposes, the pharmaceutical composition of the invention may be added as a dietary supplement. In the case of an animal, the dietary supplement may be added to the animal feed, for example, in a dose of up to about 50 grams per head per day for an adult animal (about 0.25% of their feed intake). For treatment purposes, the pharmaceutical composition of the invention may be added up to 200 grams per head per day (about 1% of their feed intake). For example, a heaped tablespoon (about 20-25 grams) can be added to the animal's daily ration of water or milk substitute.

In another of its aspects, the invention provides a dietary supplement containing enzymes and a pH buffer system. The pH buffer system is selected to causes an alkalinization of at least a portion of the gastrointestinal tract. In humans and most animals, the digestive tract outside of the abomasum or stomach, has a normal pH in the range of 6 to 6.8. Accordingly, in one embodiment of this aspect of the invention, the pH buffer system is selected to maintain a pH above 6.8, or a pH in the range of 6.8 to 7.5 in at least a portion of the gastrointestinal tract. The pH buffer system may be, for example, K₂HPO₄—KH2PO₄, Na₂HPO₄—NaH₂PO₄, calcium carbonate and calcium proprionate, sodium bicarbonate and calcium propionate, MgO and sodium bicarbonate, or MgO and calcium bicarbonate. The inventor has found that maintaining the pH of a portion of the gastrointenstinal tract above the normal pH tends to improve the activity of the added enzymes.

Thus, in one of its aspects, the present invention provides a pharmaceutical composition comprising co-particles of an adsorbent and one or more carbohydrates. The ratio of adsorbent to carbohydrates may be in a range from 1:1 to 9:1. In particular, the ratio of adsorbent to carbohydrates may be8:2. The pharmaceutical composition according to this aspect of the invention may be in a form suitable for oral consumption. The pharmaceutical composition may be in a form suitbale for administration to a non-human-animal.

The adsorbent may be selected, for example, from the group comprising active charcoal, silica-gel, zeolites, aluminum silicate, or a synthetic or naturally occurring resin. In particular, the absorbent may be active charcoal from a plant source. The one or more carbohydrates may comprise dextrose.

The pharmaceutical composition according this aspect of the invention may be used for the treatment or prophylaxis of diarrhea. The pharmaceutical composition may be used for detoxification, adsorbing digestive tract microorganisms, enhancing weight gain or for enhancing milk production in a mammal.

The invention also provides a method for treatment or prophylaxis of a gastrointestinal tract disorder of an animal comprising administering to the animal a pharmaceutical composition comprising co-particles of an adsorbent and one or more carbohydrates. The pharmaceutical composition may be administered to the animal, for example, in a dose of up to 50 grams per day, or in a dose comprising 0.25% of feed consumption of the animal. The pharmaceutical composition may also be administered to the animal in a dose of up to 200 grams per day or in a dose comprising about 1% of feed consumption of the animal. The pharmaceutical composition may be added to water or a milk substitute.

The method in accordance with this aspect of the invention may be used, for example, for the treatment or prophylaxis of diarrhea, for detoxification, or for adsorbing digestive tract microorganisms. The method may also be used for enhancing weight gain, or for enhancing milk production in a mammal.

In still another of its aspects, the invention provides a pharmaceutical composition comprising one or more enzymes and a pH buffer system, wherein the pH buffer system causes an alkalinization of at least a portion of a gastrointestinal tract. The pH buffer system maybe selected to maintain a pH above 6.8 in at least a portion of the gastrointestinal tract, or to maintain a pH in the range of 6.8 to 7.5 in at least a portion of the gastrointestinal tract.

The pH buffer system may be, for example, K₂HPO₄—KH₂PO₄, Na₂HPO₄—NaH₂PO₄, calcium carbonate and calcium proprionate, sodium bicarbonate and calcium propionate, MgO and sodium bicarbonate, or MgO and calcium bicarbonate. One or more of the enzymes may be, for example, α-amylase, bacilosine, xylanase, pectase, phytase, and cellulose.

The pharmaceutical composition in accordance with this aspect of the invention may be used for increasing milk production in a mammal or for increasing the efficiency of milk production in a mammal, where the efficiency is the ratio to daily milk production to daily feed consumption of the mammal. The mammal may be, for example, a calf, a cow or pig.

Thus, the invention also provides a method for increasing milk production in a mammal comprising administering to the mammal a pharmaceutical composition comprising one or more enzymes and a pH buffer system, wherein the pH buffer system causes an alkalinization of at least a portion of a gastrointestinal tract. The invention also provides a method for increasing the efficiency of milk production in a mammal comprising administering to the mammal a pharmaceutical composition comprising one or more enzymes and a pH buffer system, wherein the pH buffer system causes an alkalinization of at least a portion of a gastrointestinal tract, where the efficiency is the ratio to daily milk production to daily feed consumption of the mammal. The mammal may be, for example, a calf, a cow or pig.

### EXPERIMENTAL RESULTS

### Experiment 1

A study was conducted on a commercial herd of Kibbutz Gevuloth, Israel. Thirty four calves after having received the first milk (colostrums) were housed in individual hutches. The calves were divided into two groups so that the average weight of the calves and the average age of the calves were about the same for the two groups. The treatments were as follows:
1) The calves of the control group were fed a basic calf's concentrated diet including 18% crude protein, milk substitute powder with 24% crude protein mixed in water in accordance with local farm practice.
2) The calves of the treatment group were fed the same diet as the control group but supplemented with 20 g/head/day of the pharmaceutical composition of the invention having the following composition: 80% active charcoal, 20% dextrose. The composition was added to the milk substitute.

After 60 days, the calves were weighed. The results are shown in Table 1. The average daily gain in the treatment group was 5.8% higher than that of the control group. The ratio of total body gain to birth weight was 3.8% higher in the treatment group than that of the control group. Also shown in Table 1, cases of diarrhea were much lower (4 vs. 23) in the treated group in comparison with the control group. Cases requiring antibiotic or other medical treatments were also much lower in the treatment group (4 vs. 14 and 4 vs. 20).

According to the results shown in Table 1, supplementing a standard diet with the composition increased the average daily body gain by 5.8% and causes a significant reduction in the occurrence of diarrhea and other problems requiring medical treatment.

TABLE 1
Average body weight and antibiotic treatments given to calves

| | Treatment Group | Control Group |
|---|---|---|
| Initial body weight (kg) | 38.8 | 36.7 |
| Final body weight (kg) | 76.8 | 71.2 |
| Daily gain (g/day) | 620 | 586 |
| Ratio of total body gain | 0.986 | 0.950 |
| to birth weight | | |
| Cases of diarrhea | 4 | 23 |
| Cases requiring anti- | 4 | 14 |
| biotic treatment | | |
| Cases requiring other | 4 | 20 |
| medical treatments | | |

### Experiment 2

A composition of the invention comprising 70% active charcoal and 30% dextrose was tested on 120 pigs on a farm in Maipo, Chile. The animals were divided into two groups of 60 after weaning and were weighed. The treatment group received a standard animal feed supplemented with 3 grams of the pharmaceutical composition of the invention per kilo feed. The control group received the same standard feed without the composition of the invention. The pigs were 24 days old at the beginning of the experiment, and had an average initial weight of 6.5 Kg. Table 2 shows the results after 30 days. The results show that the animals of the treatment group underwent a large weight gain during the experiment than the treatment group.

TABLE 2

| | | Average body | Weight | Average body | Weight |
|---|---|---|---|---|---|
| | | weight ± S.D | gain | weight ± S.D | gain |
| | Number | after 30 days. | after 30 | after 45 days. | after 45 |
| | of Pigs | (Kg) | days | (Kg) | days |
| Control | 60 | 14.84 ± 2.32 | 8.34 | 22.75 | 16.25 |
| Group | | | | | |
| Treatment | 60 | 17.94 ± 1.62 | 11.44 | 25.65 | 19.15 |
| Group | | | | | |

### Experiment 3

Manure was collected from sows either not treated (the control group) or treated with a composition of the invention comprising 70% active charcoal and 30% dextrose (3 grams of the composition of the invention per kilogram feed added to the animal feed of the treatment group). The manure was analyzed for dry matter and nitrogen content. The results are shown in Table 3. The results show that the manure of the treated sows had less nitrogen and protein than the untreated sows, showing that the composition of the invention improves absorption of proteins and nitrogen.

TABLE 3
Manure analysis

| | Control | Treatment | |
|---|---|---|---|
| component | Group | Group | Difference |
| Day 0 | | | |
| Dry matter (% | 21.65 | 25 | |
| in fresh sample) | | | |
| Protein (% in | 24.95 | 21.31 | |
| dried sample) | | | |
| Day 4 | | | |
| Dry matter (% | 19.46 | 22.68 | |
| in fresh sample) | | | |
| Protein (% in | 22.08 | 20.73 | |
| dried sample) | | | |
| Day 18 | | | |
| Dry matter (% | 23.4 | 26.23 | |
| in fresh sample) | | | |
| Protein (% in | 23.4 | 23.36 | |
| dried sample) | | | |
| Cumulative | | | |
| Average Dry | 23.4 | 24.64 | 5.4 |
| Sample (%) | | | |
| Average | 23.4 | 21.8 | 7.5 |
| protein (%) | | | |
| Average N (%) | 3.75 | 3.49 | 7.5 |

### Experiment 4

A composition of the invention comprising active charcoal and dextrose in a ration of 7 to 3 was tested on a cow suffering from digestive tract disorders and strong diarrhea, mainly from total mix ration (TMR) diet problems, such as mycotoxins. This condition resulted in a significant reduction in milk yield from 39.5 liters/day to 31.6 liters per day over a period of 24 hours prior to administration of the composition. 0.10 full tablespoons of the composition were mixed in a bottle with 1 liter water, and administered per os to the cow. Within 48 hours after the onset of the administration of the composition, the milk production increased to 38.1 liters per day, and 24 hours later, the milk production increased to 39.8 liter per day.

### Experiment 5

An experiment was conducted over a period of about two months on three groups of calves. One group, the “charcoal” group received charcoal alone (10 grams per day per head) as a dietary supplement. A second group, the “charcoal+dextrose” group received 10 grams per day per head of a dietary supplement of the invention comprising co-particles of charcoal and dextrose in a ratio of 7:3. The calves in each group were monitored for weight gain over the course of the experiment. The results are shown in Table 4. Calves receiving the dietary supplement comprising the co-particles of charcoal and dextrose showed a daily weight gain of 0.697 kg/day that was 15.2% higher than that of the calves that received charcoal alone without any carbohydrates (0.605 kg/day). The results show that the combination of charcoal and dextrose has a synergistic effect in comparison the charcoal alone.

TABLE 4

| | | | | Average Daily |
|---|---|---|---|---|
| | Body | Body | Body | Gain in |
| | weight | weight | weight | Body weight |
| Treatment | (Start) | (end) | Gain (kg) | (kg/day) |
| Control | 55.0 | 85.7 | 30.7 | 0.570 |
| Charcoal | 52.3 | 86.0 | 33.7 | 0.605 |
| Charcoal and dextrose | 57.7 | 95.7 | 38.0 | 0.697 |

### Experiment 6

An animal feed supplement in accordance with the invention comprising lysine salts of propionic acid (propionate) in a concentration from 0.1% to 10%, sodium bicarbonate (0-55%), and the following enzymes (0.1% to 15%): α-amylase, bacilosine, xylanase, pectase, phytase, and cellulase was provided to dairy cows. The efficiency was calculated for the treatment group and the control group that did not receive the supplement, where the efficiency is defined as the ratio of the milk production (liters/day) to the daily consumption of animal feed.

Prior to the onset of the administration of the supplement, the cows had an average efficiency of about 1.6. After one month of the supplement, the efficiency of the treatment group rose to about 2. After an additional month of receiving the supplement, the efficiency of the treatment group rose to 2.5. Use of the supplement improved efficiency of feeding by reducing the amount of feed consumed by the cows while increasing the amount of milk produced without any reduction in milk solids. In addition, the use of the product reduced digestion disorders and metabolic post calving diseases such as ketosis and acidosis.

A similar experiment was conducted on two groups of 52 cows on a farm in Maipo Chile. 50 gr per cow per day of the above animal feed supplement (0.1% to 10%, sodium bicarbonate (0-55%), and the following enzymes (0.1% to 15%): α-amylase, bacilosine, xylanase, pectase, phytase, and cellulose). The experiment lasted for 30 days. At the end of the experiment, the efficiency of the treated cows was 1.85 and that of the control cows was 1.48, a percent difference of 25.07.

### Experiment 7

Cows of second milking were divided into two groups, treatment and control. Data relating to the two groups is shown in Table 5.

TABLE 5

| | Number of | Days in | Kg/day | Percent | Percent |
|---|---|---|---|---|---|
| | cows | milking | milk | fat | protein |
| Control | 79 | 180.4 | 41.93 | 3.28 | 3.25 |
| Treated | 89 | 179.1 | 41.87 | 3.24 | 3.30 |

The two groups of cows received the same diet from the same feed mixer in two feed deliveries per day. In the treated group, a dietary supplement was provided that included as a buffer system 0.6 gr sodium bicarbonate and 0.2-0.3 gr calcium propionate per kilo feed and the following enzymes (0.1% to 15%): α-amylase, bacilosine, xylanase, pectase, phytase, and cellulose. The supplement was added to the morning delivery every day immediately after delivery of the feed to the trough according to the following regimen. The buffer system was not added during weeks 1 to 3, and was included during weeks 4 and 5. Once per week the mixture was sampled for percent dry matter. Unconsumed feed was weighed once per week and was sampled for dry matter. Feed consumption (dry weight) per cow was calculated on a weekly basis by the amount of dry matter that was delivered to each group minus the weight of the unconsumed feed by the group. This difference was then divided by the number of cows in the group. Milk production was recorded weekly as the total amount of milk that each cow produced during the week throughout the experiment. A statistically significant increase in milk production was found at week 5 in the treatment group, in comparison to the control group.

### Experiment 8

An experiment was performed on cows in Piancenza, Italy. The experiment was conducted on two groups of 12 cows. The cows received a standard diet. The treated group also received a dietary supplement that included as a buffer system, 0.6 gr sodium bicarbonate and 0.2-0.3 gr calcium propionate per kilo feed mixed in the premix which was based on barley. The supplement also included the following enzymes (0.1% to 15%): α-amylase, bacilosine, xylanase, pectase, phytase, and cellulose. The control group received the premix without the supplement. The cows were allowed to adapt to the new diet for two weeks, after which the experiment commenced and the diet continued for 63 days (9 weeks). Measurements were made over a 13 week period. The results showed that the treated group had a higher efficiency, lower feed consumption, higher milk production and energy content of the milk. The treated group also showed a higher efficiency for the cheese industry, as reflected in percent protein, casein and fat content, fewer somatic cells in the milk, and curdling rate.

1.-35. (canceled)

36. A pharmaceutical composition comprising co-particles of an adsorbent and one or more carbohydrates.

37. The pharmaceutical composition according to claim 36, in a form suitable for oral consumption.

38. The pharmaceutical composition according to claim 36, wherein the adsorbent is selected from the group consisting of active charcoal, silica-gel, zeolites, aluminum silicate, a synthetic resin, and a naturally occurring resin.

39. The pharmaceutical composition according to claim 38, wherein the absorbent is active charcoal from a plant source.

40. The pharmaceutical composition according to claim 36, wherein the one or more carbohydrates comprises dextrose.

41. The pharmaceutical composition according to claim 36, wherein the ratio of adsorbent to carbohydrates is in a range of from 1:1 to 9:1.

42. The pharmaceutical composition according to claim 41, wherein the ratio of adsorbent to carbohydrates is 8:2.

43. The pharmaceutical composition according to claim 36, in a form suitable for administration to a non-human-animal.

44. A method for treatment or prophylaxis of a gastrointestinal tract disorder of an animal comprising administering to the animal a pharmaceutical composition according to claim 36.

45. The method according to claim 44, wherein the pharmaceutical composition is administered to the animal in a dose of up to 50 grams per day.

46. The method according to claim 44, wherein the pharmaceutical composition is administered in a dose comprising 0.25% of feed consumption of the animal.

47. The method according to claim 44, wherein the pharmaceutical composition is administered to the animal in a dose of up to 200 grams per day.

48. The method according to claim 44, wherein the pharmaceutical composition is administered in a dose comprising about 1% of feed consumption of the animal.

49. The method according to claim 44, wherein the pharmaceutical composition is added to water or milk substitute.

50. The method according to claim 44, for the treatment or prophylaxis of diarrhea.

51. The method according to claim 44, for detoxification.

52. The method according to claim 44, for adsorbing digestive tract microorganisms.

53. The method according to claim 44, for enhancing weight gain.

54. The method according to claim 44, for enhancing milk production in a mammal.

55. A pharmaceutical composition comprising one or more enzymes and a pH buffer system, wherein the pH buffer system causes an alkalinization of at least a portion of a gastrointestinal tract.

56. The pharmaceutical composition according to claim 55, wherein the pH buffer system maintains a pH above 6.8 in at least a portion of the gastrointestinal tract.

57. The pharmaceutical composition according to claim 55, wherein the pH buffer system maintains a pH in the range of from 6.8 to 7.5 in at least a portion of the gastrointestinal tract.

58. The pharmaceutical composition according to claim 55, wherein the pH buffer system is selected from the group consisting of K₂HPO₄—KH2PO₄, Na₂HPO₄—NaH₂PO₄, calcium carbonate and calcium proprionate, sodium bicarbonate and calcium propionate, MgO and sodium bicarbonate, and MgO and calcium bicarbonate.

59. The pharmaceutical composition according to claim 55, wherein one or more of the enzymes are selected from the group consisting of α-amylase, bacilosine, xylanase, pectase, phytase, and cellulose.

60. A method for increasing milk production in a mammal comprising administering to the mammal a pharmaceutical composition according to claim 55.

61. A method for increasing the efficiency of milk production in a mammal comprising administering to the mammal a pharmaceutical composition according to claim 55, the efficiency being the ratio of daily milk production to daily feed consumption of the mammal.

62. The method according to claim 60, wherein the mammal is a cow or pig.

63. The method according to claim 61, wherein the mammal is a cow or pig.
